(19) United States
(12) Patent Application Publication
(10) Pub. No.: US 2018/0296579 A1
(43) Pub. Date: Oct. 18, 2018

(54) METHODS OF TREATING IBRUTINIB-RESISTANT DISEASE

(71) Applicant: Rigel Pharmaceuticals, Inc., South San Francisco, CA (US)

(72) Inventors: Yasumichi Hitoshi, Brisbane, CA (US); Nan Lin, Redwood City, CA (US); Rajinder Singh, Belmont, CA (US)

(21) Appl. No.: 15/568,206

(22) PCT Filed: Apr. 18, 2016
(86) PCT No.: PCT/US2016/028134

§ 371 Date: Oct. 20, 2017

Related U.S. Application Data
(60) Provisional application No. 62/152,481, filed on Apr. 24, 2015.

(51) Int. Cl. A61K 31/675; A61K 45/06; C12Q 1/6886

(52) U.S. Cl. CPC A61K 31/675; C12Q 2600/106; C12Q 1/6886; A61K 45/06

(57) ABSTRACT

Disclosed are methods of treating an ibrutinib-resistant disease in a mammal with a compound of Formula (I): wherein R is described herein. In certain embodiments, a compound of Formula (I) inhibits the activity of a variant Btk, providing a method of treating ibrutinib-resistant diseases, such as ibrutinib-resistant lymphoma.

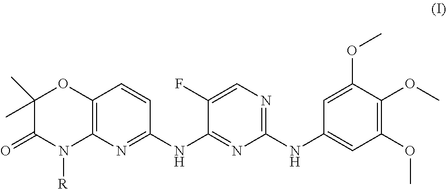

### FIELD OF INVENTION

This invention relates to the treatment of ibrutinib-resistant disease with Syk kinase inhibitors. This invention relates more particularly to the field of treating ibrutinib-resistant diseases, particularly cancer, and more particularly lymphoma.

### TECHNICAL BACKGROUND

Bruton's tyrosine kinase (abbreviated Btk or BTK and also known as tyrosine-protein kinase Btk) is an enzyme that is encoded by the Btk gene of the mammalian X chromosome. Btk plays a crucial role in B-cell development.

At least 400 mutations of the Btk gene have been identified. Mutations in the Btk gene (i.e., variant Btk) are implicated in many diseases. For example, variant Btk are associated with the primary immunodeficiency disease X-linked agammaglobulinemia (Bruton's agammaglobulinemia).

Ibrutinib (1-[(3R)-3-[4-Amino-3-(4-phenoxyphenyl)-1H-pyrazolo[3,4-d]pyrimidin-1-yl]piperidin-1-yl]prop-2-en-1-one), also known as PCI-32765 and marketed under the name Imbruvica®) is an anticancer drug targeting B-cell malignancies. Ibrutinib is a Btk kinase inhibitor whose mechanism of action relies on covalent reaction with Cys481 in the Btk ATP-pocket.

Mutations of Btk can lead to ibrutinib-resistant diseases. For example, in ibrutinib-resistant cancers, the Cys481 is mutated to a Ser481 (C481S). Because of the decreased nucleophilicity of Ser481 in the C481S variant Btk, ibrutinib binds with less affinity and in a less desirable conformation than with the wild-type Btk. For example, Cheng et al. suggest that ibrutinib binds to the C481S with a 500-fold lower affinity in cell culture (Cheng, S. et al., Leukemia 2015, 29, 895-900). As a result, ibrutinib is no longer active against such variants.

### SUMMARY

We have found that Syk inhibitors, like ibrutinib and other Btk inhibitors, bind to Btk, but without relying on a covalent interaction with Cys481. Therefore, Syk inhibitors may be useful in the treatment of ibrutinib-resistant diseases.

We recognized that new therapeutic agents that inhibit the activity of ibrutinib-resistant kinases are useful for treating human or animal disorders in which ibrutinib-resistance is implicated. In one aspect, the ibrutinib-resistant kinases may be implicated in cancer, for example, ibrutinib-resistant lymphoma.

Accordingly, the present disclosure provides a method of treating an ibrutinib-resistant disease in a mammal by administering a compound of Formula (I):

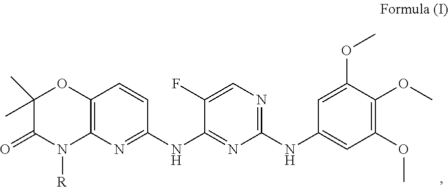

wherein R is defined below.

Another aspect of the present disclosure comprises methods for treating an ibrutinib-resistant disease in a mammal that has an ibrutinib resistance-conferring mutation of an enzyme that mediates growth and/or proliferation of the disease by administering to the mammal an effective amount of a compound of Formula (I).

Another aspect of the disclosure comprises inhibiting a variant Btk by contacting the Btk with an effective amount of a compound of Formula (I).

Another aspect of the disclosure comprises identifying a mammal with an ibrutinib-resistant disease and administering an effective amount of a compound of Formula (I) to the mammal.

All publications referenced herein are incorporated by reference in their entirety to the extent they are not inconsistent with the teachings presented herein.

### DETAILED DESCRIPTION

In one aspect, the present disclosure provides a method of treating an ibrutinib-resistant disease in a mammal, the method comprising administering an effective amount of a Syk inhibitor to the mammal.

In some embodiments of the method, the Syk inhibitor is a compound of Formula (I):

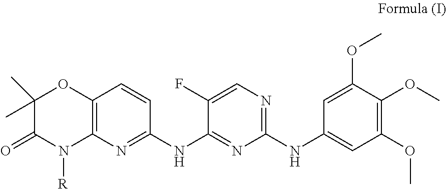

wherein R is hydrogen, —CH₂OP(O)(O⁻)₂X⁺₂, or —CH₂OP(O)(O⁻)₂Y²⁻,
and each X is independently a hydrogen ion or a monovalent cation, and Y²⁻ is a divalent cation;

or a pharmaceutically acceptable salt, hydrate or solvate thereof.

In some examples, the compound of Formula (I) is a hydrate or solvate. For example, a hexahydrate.

In some embodiments, the compound of Formula (I) may be of Formula (Ia):

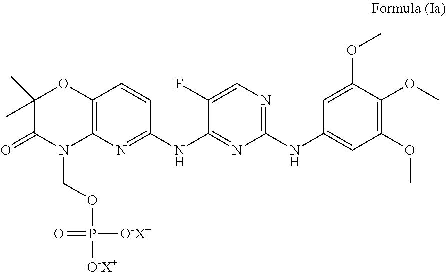

wherein each X⁻ is an alkali metal cation, such as sodium (Na⁻), potassium (K⁻), or lithium (Li³¹ ).

In some embodiments, the compound of Formula (Ia) is in the form of Compound (I)

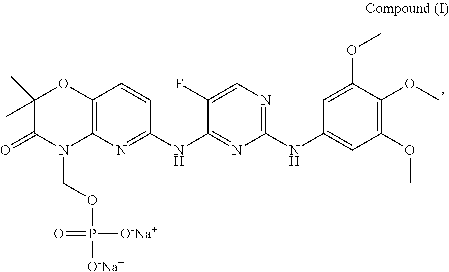

or a hydrate or solvate thereof. Compound (I) is disclosed in international patent application WO2006/078846 as (6-(5-fluoro-2-(3,4,5-trimethoxyphenylamino)pyrimidin-4-ylamino)-2,2-dimethyl-3-oxo-2H-pyrido[3,2-b][1,4]oxazin-4(3H)-yl)methyl phosphate disodium salt.

In another embodiment, Formula (Ia) is in the hexahydrate form of Compound (Ia):

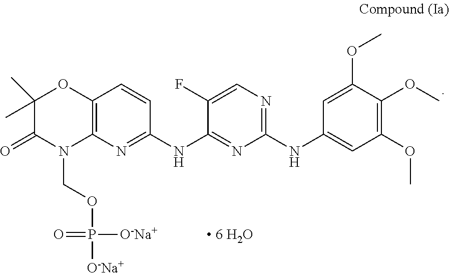

In some embodiments, the compound of Formula (I) may be of Formula (Ib):

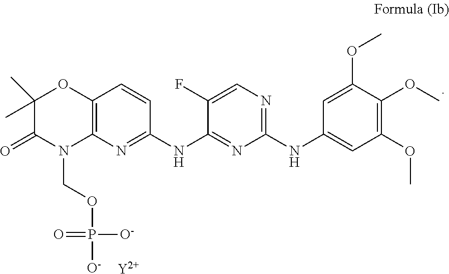

wherein the divalent cation Y²⁺ is an alkali earth metal cation, such as a magnesium (Mg²⁺), calcium (Ca²⁺) or barium (Ba²⁺).

In some embodiments, the compound of Formula (I) is Compound (II):

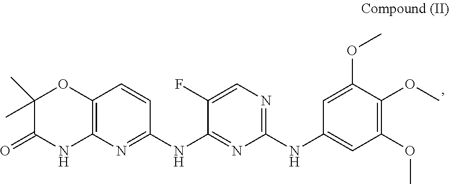 [SMILES: COc1cc(Nc2ncc(F)c(Nc3ccc4c(n3)NC(=O)C(C)(C)O4)n2)cc(OC)c1OC]

or a pharmaceutically acceptable salt, hydrate or solvate thereof. Compound (II) is disclosed in international patent application WO2005/016893 and in WO2006/078846 as N4-(2,2-dimethyl-3-oxo-4H-5-pyrido[1,4]oxazin-6-yl)-5-fluoro-N2-(3,4,5-trimethoxyphenyl)-2,4-pyrimidinediamme.

In another aspect, the present disclosure provides pharmaceutical compositions comprising a compound of Formula (I) and a pharmaceutically acceptable excipient. For example, such compositions include hydrolytically stable pharmaceutical formulations of Compound (I) and a water sequestering agent prepared by a wet granulation process (international patent application WO 2009/061909). Other suitable pharmaceutical compositions are disclosed in WO 2013/014454.

In some embodiments of the method, administration of the compound of Formula (I) is performed in conjunction with administration of a second Syk inhibitor, concurrently or in series. The second inhibitor may be a different compound of Formula (I) or any Syk inhibitor known in the art.

In another aspect, the present disclosure provides methods for treating an ibrutinib-resistant disease in a mammal that has an ibrutinib resistance-conferring mutation of an enzyme that mediates growth and/or proliferation of the disease, the method comprising administering an effective amount of a compound of Formula (I) to the mammal. In some embodiments, the ibrutinib resistance-conferring mutation is of Btk. The mutation may be of the Cys481 residue, particularly a C481S mutation.

In some embodiments the ibrutinib-resistant disease is an ibrutinib-resistant cancer. The cancer may one that affects hematopoietic and/or lymphoid tissues, such as lymphoma. In some embodiments, the ibrutinib-resistant lymphoma is chronic lymphocytic leukemia (CLL), mantle cell lymphoma, Waldenstrom's macroglobulinemia, diffuse large B-cell lymphoma (DLBCL), follicular lymphoma, marginal zone lymphoma, multiple myeloma, acute myeloid leukemia (AML), or acute lymphoblastic leukemia (ALL).

In some embodiments, the ibrutinib resistance-conferring mutation is non-Btk. For example, mutations to PLCG2 (1-Phosphatidylinositol-4,5-bisphosphate phosphodiesterase gamma-2) have also been found to reduce ibrutinib sensitivity in vitro (“Recurrent BTK and PLCG2 Mutations Confer Ibrutinib Resistance” Cancer Discovery 2014; 4:866). Other mutations implicated in conferring ibrutinib-resistance are also known in the art and can therefore be treated by administering a compound of Formula (I). To avoid doubt, unless otherwise stated, all references to uses of a compound of Formula (I) include the use of any of the species and subgenera of Formula (I) disclosed herein.

Another aspect of the disclosure comprises inhibiting a variant Btk by contacting the variant Btk with an effective amount of a compound of Formula (I). In some embodiments, the variant Btk is a Cys481 variant, such as Btk with a C481S mutation.

In some embodiments, the variant Btk is contacted in a cell, such as a B-cell. The cell may be a cell implicated in a disease, such as a cancer cell. For example, the cell may be a lymphoma cell. In other examples, the cell may be a cell of chronic lymphocytic leukemia (CLL), mantle cell lymphoma, Waldenstrom's macroglobulinemia, diffuse large B-cell lymphoma (DLBCL), follicular lymphoma, marginal zone lymphoma, multiple myeloma, acute myeloid leukemia (AML), or acute lymphoblastic leukemia (ALL).

In some embodiments, the variant Btk is contacted with a second Syk inhibitor, concurrently or in series. The second inhibitor may be a different compound of Formula (I) or any Syk inhibitor known in the art.

Another aspect of the disclosure comprises identifying a mammal with an ibrutinib-resistant disease and administering an effective amount of a Syk inhibitor to the mammal. The Syk inhibitor may be any compound of Formula (I) as described herein and the ibrutinib-resistant disease may be as describe herein.

Identifying a mammal with an ibrutinib-resistant disease may be accomplished by any number of methods known in the art. For examples, the identifying may be done by DNA sequencing, particularly whole-exome sequencing. In embodiments where the ibrutinib-resistant disease is caused by an ibrutinib resistance-conferring mutation to Btk, that mutation may be identified prior to the administration of a compound of Formula (I). For example, a C481S mutation in Btk may be identified by DNA sequencing. In embodiments where the ibrutinib-resistant disease is caused by an ibrutinib resistance-conferring mutation to an enzyme other than Btk, that mutation may be identified prior to the administration of a compound of Formula (I). For example, the mutations to R665W and L845F PLCG2 may be identified by DNA sequencing. See Woyach et al., “Resistance Mechanisms for the Bruton's Tyrosine Kinase Inhibitor Ibrutinib” N Engl J Med 2014; 370:2286-94.

In another aspect, the disclosure comprises combination therapies for the treatment of cancer, including both pre-malignant and malignant neoplasms. In this aspect, the disclosure comprises a method of treating cancer comprising administering to a subject a compound of Formula (I) in conjunction with a therapeutic treatment of cancer. (Unless stated otherwise, compounds and methods used in conjunction with treatment with Formula (I) can be employed concurrently or serially with treatment with Formula (I).) In some embodiments of the disclosure, the compound of Formula (I) is used in combination with standard-of-care anti-proliferative treatments of cancer. For example, the method can involve the administration of a compound of Formula (I) and radiation and/or surgical tumor removal. Efficacy of treatment can be determined by any art recognized method generally employed for the particular cancer being treated and includes, for example, retardation, inhibition, or regression of tumor growth.

One embodiment of treating cancer in a subject comprises administering to a subject in need thereof an effective amount of a compound of Formula (I) in combination with the administration of a therapeutically effective amount of one or more chemotherapeutic agents, wherein the one or more chemotherapeutic agents is selected from the group consisting of antimetabolites, alkylating agents, coordination compounds, platinum complexes, DNA cross-linking compounds, inhibitors of transcription enzymes, tyrosine kinase inhibitors, protein kinase inhibitors, topoisomerase inhibitors, DNA minor-groove binding compounds, vinca alkyloids, taxanes, antitumor antibiotics, hormones, aromatase inhibitors, enzymes, growth factor receptors antibodies, cytokines, cell surface markers antibodies, HDAC inhibitors, HSP 90 inhibitors, BCL-2 inhibitors, B-raf inhibitors, MEK inhibitors, mTOR inhibitors, proteasome inhibitors and monoclonal antibodies.

Another embodiment of methods for treating a subject comprises administering to the subject an effective amount of a compound of Formula (I) in combination with the administration of a therapeutically effective amount of one or more chemotherapeutic agents, the one or more chemotherapeutic agents being independently selected from the group consisting of mechlorothamine, cyclophosphamide, ifosfamide, melphalan, chlorambucil, ethyleneimines, methylmelamines, procarbazine, dacarbazine, temozolomide, busulfan, carmustine, lomustine, methotrexate, fluorouracil, capecitabine, cytarabine, gemcitabine, cytosine arabinoside, mecaptopurine, fludarabine, cladribine, thioguanine, azathioprine, vinblastine, vincristine, paclitaxel, docetaxel, colchicine, actinomycin D, daunorubicin, bleomycin,L-asparaginase, cisplatin, carboplatin, oxaliplatin, prednisone, dexamethasone, amino glutethimide, formestane, anastrozole, hydroxyprogesterone caproate, medroxyprogesterone, tamoxifen, amsacrine, mitoxantrone, topotecan, irinotecan, camptothecin, afatinib, axitinib, bosutinib, bortezomib, carfilzomib, cabozantinib, cediranib, crizotinib, dasatinib, dabrafenib, evorolimus, ibrutinib, LDK378, LGX818, MEK162, regorafenib, ruxolitinib, selumetinib, sorafenib, trametinib, vemurafenib, erlotinib, gefitinib, idelalasib, imatinib, lapatinib, lestaurtinib, nilotinib, palbociclib, pazopanib, pomatinib, semaxanib, sirolimus, sunitinib, temsirolimus, vatalanib, vandetanib, anti Her2 antibodies, interferon-α, interferon-γ, interleukin 2, GM CSF, anti CTLA 4 antibodies, rituximab, anti CD33 antibodies, MGCD0103, vorinostat, 17-AAG, thalidomide, lenalidomide, rapamycin, CCI-779, doxorubicine, gemcitabine, melphalan, NPI052, gemtuzumab, alemtuzumab, cetuximab, ibritumomab tiuxaetan, tositumomab, iodine-131 tositumomab, trastuzumab, ado-trastuzumab emtansine, obinutuzumab, bevacizumab, rituximab, and anti-TRAIL death receptor antibodies.

Other chemotherapeutic agents for combination with a compound of Formula (I) include checkpoint pathway inhibitors, such as PD-1 inhibitors (e.g., nivolumab and lambrolizumab) and PD-L1 inhibitors (e.g., pembrolizumab, MEDI-4736 and MPDL3280A/RG7446). Additional checkpoint inhibitors for combination with the compounds disclosed herein include Anti-LAG-3 agents, such as BMS-986016 (MDX-1408). Further chemotherapeutic agents for combination with a compound of Formula (I) include Anti-SLAMF7 agents (e.g., the humanized monoclonal antibody elotuzumab (BMS-901608)), anti-KIR agents (e.g., the anti-KIR monoclonal antibody lirilumab (BMS-986015)), and anti-CD137 agents (e.g., the fully human monoclonal antibody urelumab (BMS-663513)).

### Definitions

The compounds disclosed herein can also be provided as pharmaceutically acceptable salts. The term “pharmaceutically acceptable salts” or “a pharmaceutically acceptable salt thereof” refer to salts prepared from pharmaceutically acceptable non-toxic acids or bases including inorganic acids and bases and organic acids and bases. If the compound is basic, salts may be prepared from pharmaceutically acceptable non-toxic acids. Such salts may be, for example, acid addition salts of at least one of the following acids: benzenesulfonic acid, citric acid, α-glucoheptonic acid, D-gluconic acid, glycolic acid, lactic acid, malic acid, malonic acid, mandelic acid, phosphoric acid, propanoic acid, succinic acid, sulfuric acid, tartaric acid (d, l, or dl), tosic acid (toluenesulfonic acid), valeric acid, palmitic acid, pamoic acid, sebacic acid, stearic acid, lauric acid, acetic acid, adipic acid, carbonic acid, 4-chlorobenzenesulfonic acid, ethanedisulfonic acid, ethylsuccinic acid, fumaric acid, galactaric acid (mucic acid), D-glucuronic acid, 2-oxo-glutaric acid, glycerophosphoric acid, hippuric acid, isethionic acid (ethanolsulfonic acid), lactobionic acid, maleic acid, 1,5-naphthalene-disulfonic acid, 2-naphthalene-sulfonic acid, pivalic acid, terephthalic acid, thiocyanic acid, cholic acid, n-dodecyl sulfate, 3-hydroxy-2-naphthoic acid, 1-hydroxy-2-naphthoic acid, oleic acid, undecylenic acid, ascorbic acid, (+)-camphoric acid, d-camphorsulfonic acid, dichloroacetic acid, ethanesulfonic acid, formic acid, hydriodic acid, hydrobromic acid, hydrochloric acid, methanesulfonic acid, nicotinic acid, nitric acid, orotic acid, oxalic acid, picric acid, L-pyroglutamic acid, saccharine, salicylic acid, gentisic acid, and/or 4-acetamidobenzoic acid.

The compounds described herein can also be provided in prodrug form. “Prodrug” refers to a derivative of an active compound (drug) that undergoes a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety can proceed spontaneously, such as by way of a hydrolysis reaction, or it can be catalyzed or induced by another agent, such as by an enzyme, by light, by acid, or by a change of or exposure to a physical or environmental parameter, such as a change of temperature. The agent can be endogenous to the conditions of use, such as an enzyme present in the cells to which the prodrug is administered or the acidic conditions of the stomach, or it can be supplied exogenously. A wide variety of progroups, as well as the resultant promoieties, suitable for masking functional groups in the active drugs to yield prodrugs are well known in the art. For example, a hydroxyl functional group can be masked as a sulfonate, ester or carbonate promoiety, which can be hydrolyzed in vivo to provide the hydroxyl group. An amino functional group can be masked as an amide, carbamate, imine, urea, phosphenyl, phosphoryl or sulfenyl promoiety, which can be hydrolyzed in vivo to provide the amino group. A carboxyl group can be masked as an ester (including silyl esters and thioesters), amide or hydrazide promoiety, which can be hydrolyzed in vivo to provide the carboxyl group. Specific examples of suitable progroups and their respective promoieties will be apparent to those of skill in the art.

The compounds disclosed herein can also be provided as N-oxides.

The presently disclosed compounds, salts, prodrugs and N-oxides can be provided, for example, in solvate or hydrate form.

As used herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

One of ordinary skill in the art of medicinal chemistry also will appreciate that the disclosed structures are intended to include isotopically enriched forms of the present compounds. As used herein “isotopes” includes those atoms having the same atomic number but different mass numbers. As is known to those of skill in the art, certain atoms, such as hydrogen occur in different isotopic forms. For example, hydrogen includes three isotopic forms, protium, deuterium and tritium. As will be apparent to those of skill in the art upon consideration of the present compounds, certain compounds can be enriched at a given position with a particular isotope of the atom at that position. For example, compounds having a fluorine atom, may be synthesized in a form enriched in the radioactive fluorine isotope ¹⁸F. Similarly, compounds may be enriched in the heavy isotopes of hydrogen: deuterium and tritium; and similarly can be enriched in a radioactive isotope of carbon, such as ¹³C. Such isotopic variant compounds undergo different metabolic pathways and can be useful, for example, in studying the ubiquitination pathway and its role in disease.

Reference to “combination therapy” and treatment with a compound of Formula (I) “in conjunction with” another therapeutic treatment means that the compound and other therapeutic treatment can be administered simultaneously or sequentially such that the resultant treatment is more efficacious than either treatment alone.

As used herein, the term “mammal” is intended to include, but not be limited to, humans, pigs, cattle, cats, dogs and rodents.

As used herein, the term “cell” is intended to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” an enzyme with a compound includes the administration of a compound described herein to an individual or patient, such as a human, as well as, for example, introducing a compound into a sample containing a cellular or purified preparation containing the enzyme.

As used herein, the terms “individual,” “patient,” or “subject” are used interchangeably, refers to any animal, including mammals, preferably mice, rats, other rodents, rabbits, dogs, cats, swine, cattle, sheep, horses, or primates, and most preferably humans.

As used herein, the terms “catalytic pocket”, “catalytic site”, “active site” collectively and indistinctly refer to a region of the enzyme that contains amino acid residues responsible for the substrate binding (charge, hydrophobicity, steric hindrance) and catalytic amino acid residues which act as proton donors or acceptors or are responsible for binding a cofactor and participate in the catalysis of a chemical reaction.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

In certain embodiments, a therapeutically effective amount is an amount suitable for the stated effect, such as,
(1) preventing a disease; for example, preventing a disease, condition or disorder in an individual who may be predisposed or otherwise susceptible to the disease, condition or disorder but does not yet experience or display the pathology or symptomatology of the disease;
(2) treating a disease, which means
   (a) inhibiting the disease, such as, for example, by inhibiting the disease (or a condition or disorder thereof) in an individual who is experiencing or displaying the pathology or symptomatology of the disease, condition or, disorder, or
   (b) ameliorating the disease (including a symptom or symptoms thereof) by for example, ameliorating the disease (or a condition or disorder thereof) in an individual who is experiencing or displaying the pathology or symptomatology of the disease, condition or disorder (i.e., reversing the pathology and/or symptomatology), such as decreasing the severity of disease; or
(3) eliciting the referenced biological effect (e.g., inhibition of a variant Btk).

Manifestation of amelioration of a disease condition may require the concomitant or sequential administration of additional therapeutic agents, such as antineoplastic agents in the case of cancer, or antiretroviral agents in the case of viral diseases. For example, administration of Btk variant inhibitors for the treatment of cancer does not always produce a direct antitumor effect when used as a single agent. But when combined with chemotherapeutic drugs (antineoplastic), the antitumor effect observed is higher than the sum of effects of each agent alone. In one embodiment the present compounds are used as immunomodulators to increase an immune response or to abrogate a tumor's ability to evade the immune response. In one embodiment of a method for using the present compounds, one or more Syk inhibitor is used in combination with an immuno-oncology treatment.

### Pharmaceutical Formulations and Dosage Forms

The compounds of Formula (I) can be administered, for example, orally, topically, parenterally, by inhalation or spray, or rectally in dosage unit formulations containing one or more pharmaceutically acceptable carriers, diluents or excipients. The term parenteral as used herein includes percutaneous, subcutaneous, intravascular (e.g., intravenous), intramuscular, and intrathecal injection or infusion techniques and the like.

Pharmaceutical compositions can be made using the presently disclosed compounds. For example, in one embodiment, a pharmaceutical composition includes a pharmaceutically acceptable carrier, diluent or excipient, and compound of Formula (I), such as the wet granulated formulations described in international patent application WO2009/061909. Other suitable formulations for use in the present methods include those described in WO2013/014454.

In the pharmaceutical compositions disclosed herein, one or more compounds of Formula (I) may be present in association with one or more pharmaceutically acceptable carriers, diluents or excipients, and, if desired, other active ingredients. The pharmaceutical compositions containing compounds of Formula (I) may be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any suitable method for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by suitable techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use can also be presented as lozenges.

Aqueous suspensions contain the active materials in admixture with excipients suitable for the manufacture of aqueous suspensions. Such excipients can be suspending agents, for example sodium carboxymethylcellulose, methylcellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

In some embodiments, the pharmaceutically acceptable carrier, diluent, or excipient is not water. In other embodiments, the water comprises less than 50% of the composition. In some embodiments, compositions comprising less than 50% water have at least 1%, 2%, 3%, 4% or 5% water. In other embodiments, the water content is present in the composition in a trace amount.

In some embodiments, the pharmaceutically acceptable carrier, diluent, or excipient is not alcohol. In other embodiments, the alcohol comprises less than 50% of the composition. In some embodiments, compositions comprising less than 50% alcohol have at least 1%, 2%, 3%, 4% or 5% alcohol. In other embodiments, the alcohol content is present in the composition in a trace amount.

Syrups and elixirs can be formulated with sweetening agents, for example glycerol, propylene glycol, sorbitol, glucose or sucrose. Such formulations can also contain a demulcent, a preservative, flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils can be employed as a solvent or suspending medium. For this purpose any bland fixed oil can be employed including synthetic mono- or diglycerides. In addition, fatty acids such as oleic acid find use in the preparation of injectables.

Compounds of Formula (I) can also be administered in the form of suppositories, e.g., for rectal administration of the drug. These compositions can be prepared by mixing the compound with a suitable non-irritating excipient that is solid at ordinary temperatures but liquid at the rectal temperature and will therefore melt in the rectum to release the drug. Such materials include cocoa butter and polyethylene glycols.

Compounds of Formula (I) can also be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The compositions can be formulated in a unit dosage form, each dosage containing from about 25 to about 250 mg, more usually about 50 to about 150 mg or from about 100 to about 200 mg, of the compound of Formula I.

In one embodiment, a unit dosage form comprises greater than or equal to 60 mg of Formula (I) and/or hydrate thereof (for example 60 mg, 70 mg, 80 mg, 90 mg, 100 mg, 110 mg, 120 mg, 130 mg, 140 mg, 150 mg, 160 mg, 170 mg, 180 mg, 190 mg or 200 mg) and an amount of one or more effervescent agents that is sufficient to provide satisfactory in vitro dissolution; and further comprising one or more pharmaceutically acceptable ingredients. For the avoidance of doubt, each of the previous integers represents a separate and independent aspect of the invention.

In another aspect of the invention a unit dosage form of the pharmaceutical composition comprises between about 60 mg to about 300 mg of Formula (I) and/or hydrate thereof.

In another aspect of the invention a unit dosage form of the pharmaceutical composition comprises between about 60 mg to about 250 mg of Formula (I) and/or hydrate thereof.

In a still further aspect, a unit dosage form of the pharmaceutical composition comprises between about 100 mg to about 200 mg of Formula (I) and/or hydrate thereof.

In a yet further aspect, a unit dosage form of the pharmaceutical composition comprises between about 125 mg to about 190 mg of Formula (I) and/or hydrate thereof.

In a specific aspect of the invention, a unit dosage form of the pharmaceutical composition comprises 63 mg±3 mg of Formula (I) and/or hydrate thereof.

In a specific aspect of the invention, a unit dosage form of the pharmaceutical composition comprises 126 mg±13 mg of Formula (I) and/or hydrate thereof.

In a further specific aspect of the invention, a unit dosage form of the pharmaceutical. As used herein, “dosage strength” is the equivalent mass of the free acid form of Compound I based on the amount of Compound (Ia) present in the dosage form, which may be, by way of example a tablet or a capsule. Thus, a dosage strength of 50 mg will contain about 63 mg of Compound (Ia). Specific dosage strengths for use herein are about 50 mg, about 100 mg, about 150 mg, about 200 mg, and about 250 mg.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of a compound described herein.

The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. For example, the compounds described herein can be provided in an aqueous physiological buffer solution containing about 0.1 to about 10% w/v of the compound for parenteral administration. Some typical dose ranges are from about 1μg/kg to about 1 g/kg of body weight per day. In some embodiments, the dose range is from about 0.01 mg/kg to about 100 mg/kg of body weight per day. More typically, the therapeutic dosage of compounds of Formula I, such as Compounds I and II is between about 50 mg and 800 mg administered over one, two or three dosages per day. In one embodiment, a therapeutic dosage is from about 100 mg twice a day to about 300 mg twice a day, such as from about a 150 mg dosage strength administered twice a day up to about a 250 mg dosage strength twice daily. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds described herein can also be formulated in combination with one or more additional active ingredients which can include any pharmaceutical agent such as anti-viral agents, vaccines, antibodies, immune enhancers, immune suppressants, anti-inflammatory agents and the like.

### EXAMPLES

### X-Ray Crystal Structure Analysis

A comparison of co-crystal structures of Ibrutinib in Btk and Compound (II) in Syk, wild-type Btk, and variant Btk was performed

Ibrutinib in Btk—Ibrutinib docks inside Btk and forms a covalent bond to Cys481. The electrophilic moiety of Ibrutinib is arranged close to the nucleophilic Cys481 by reversibly binding. The electrophilic group is at a close distance (1.5-2 Å) and in an orientation that is highly favorable for reaction.

Compound (II) in Syk and wild-type Btk—Without limitation to any particular theory, it is believed that Compound (II) binds in the same conformation in Btk and Syk, but the binding in Btk is not reliant on the Cys481 interaction. The trimethoxyphenyl group of Compound (II) has a hydrogen bond with the LSY458 of Syk. A comparable hydrogen bond is not available in Btk, which has a much smaller amino acid (ASN484) in the same position as the LSY458 of Syk. This hydrogen bond difference likely contributes to potency difference of Compound (II) in Syk and Btk. Syk also has a PRO455 in the same position as the Cys481 of Btk, but Compound (II) has limited interaction with both residues. In Btk, the primary interaction of the trimethoxyphenyl group is with the LSY458. In Btk, the trimethoxyphenyl group of Compound (II) is relatively far from the Cys481; approximately 5 Å.

Compound (II) in variant Btk—Ser is a weaker nucleophile for Michael addition than Cys. With the C481S mutation, the variant Btk does not retain its covalent binding capability. Based on the X-ray crystallographic data, however, Compound (II), whose binding does not depend on Cys481, appears to bind the wild-type Btk and the variant Btk the same. From docking studies using the X-ray crystallographic data, Compound (II) is expected to have a similar potency of inhibition for both the wild-type and variant Btk.

### Btk Inhibition Assay

Reagent: Base Reaction buffer; 20 mM Hepes (pH 7.5), 10 mM MgCl_{2,}1 mM EGTA, 0.02% Brij35, 0.02 mg/ml BSA, 0.1 mM Na₃VO₄, 2 mM DTT, 1% DMSO Required cofactors are added individually to each kinase reaction.

Compound handling: Testing compounds were dissolved in 100% DMSO to specific concentration. The serial dilution was conducted by epMotion 5070 in DMSO.

Reaction Procedure:
1. Prepare substrate in freshly prepared Reaction Buffer
2. Deliver any required cofactors to the substrate solution above
3. Deliver kinase into the substrate solution and gently mix
4. Deliver compounds in 100% DMSO into the kinase reaction mixture by Acoustic technology (Echo550; nanoliter range), incubate for 20 min at room temp
5. Deliver ₃₃P-ATP (Specific activity 10 □Ci/□01) into the reaction mixture to initiate the reaction
6. Incubate for 2 hours at room temperature
7. Detect kinase activity by filter-binding method

Compound (II) inhibited C481 S variant Btk with an IC₅₀ of less than 100 nM, and wild-type Btk with an IC₅₀ of about 140 nM.

1. A method of treating an ibrutinib-resistant disease in a mammal, the method comprising administering an effective amount of a compound of Formula (I): 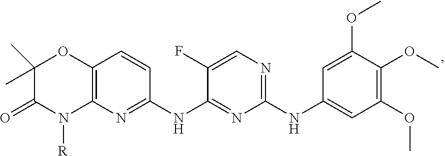
wherein R is hydrogen, —CH₂OP(O)(O⁻)₂X⁻_{2,} or —CH₂OP(O)(O⁻)₂Y²⁺, and each X is independently a hydrogen ion or a monovalent cation, and Y²⁺ is a divalent cation;
or a pharmaceutically acceptable salt, hydrate or solvate thereof;
to the mammal.

2. The method of claim 1, wherein the mammal has an ibrutinib resistance-conferring mutation of an enzyme that mediates growth and/or proliferation of the disease.

3. The method of claim 2, wherein the ibrutinib resistance-conferring mutation is of Btk.

4. The method of claim 3, wherein the mutation of the Btk is of the Cys481 residue.

5. The method of claim 4, wherein the mutation is C481S.

6. The method of claim 1, wherein the ibrutinib-resistant disease is ibrutinib-resistant cancer.

7. The method of claim 6, wherein the ibrutinib-resistant cancer is ibrutinib-resistant lymphoma.

8. The method of claim 7, wherein the ibrutinib-resistant lymphoma is chronic lymphocytic leukemia (CLL), mantle cell lymphoma, Waldenstrom's macroglobulinemia, diffuse large B-cell lymphoma (DLBCL), follicular lymphoma, marginal zone lymphoma, multiple myeloma, acute myeloid leukemia (AML), or acute lymphoblastic leukemia (ALL).

9. The method of claim 1, wherein the compound is of Formula (Ia): 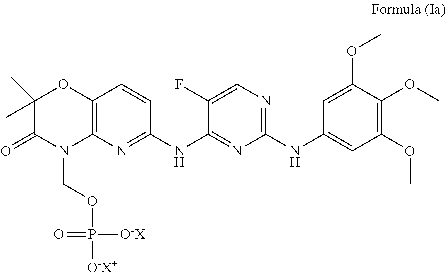
wherein each X⁺is an alkali metal cation;
or a hydrate or solvate thereof.

10. The method of claim 9, wherein the compound is Compound (I): 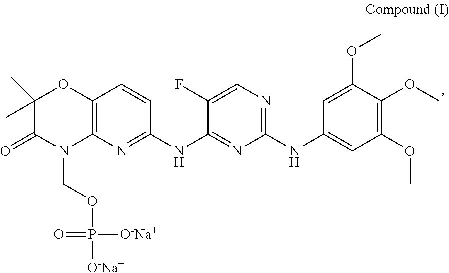
or a hydrate or solvate thereof.

11. The method of claim 1, wherein the compound is of Formula (Ib): 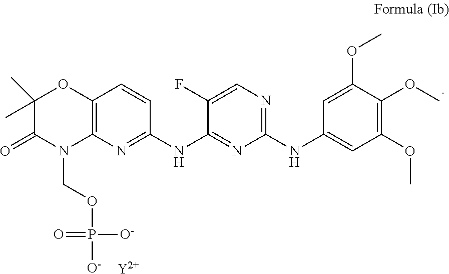
wherein the divalent cation Y²⁻ is an alkali earth metal;
or a hydrate or solvate thereof.

12. The method of claim 1, wherein the compound is Compound (II): 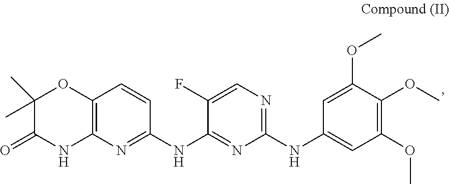 [SMILES: COc1cc(Nc2ncc(F)c(Nc3ccc4c(n3)NC(=O)C(C)(C)O4)n2)cc(OC)c1OC]
or a pharmaceutically acceptable salt, hydrate or solvate thereof.

13. The method of claim 1, further comprising the administration of a second Syk inhibitor.

14. The method of claim 1, further comprising first identifying a mammal with an ibrutinib-resistant disease.

15. The method of claim 14, wherein the identifying comprises DNA sequencing.

16. The method of claim 15, wherein the DNA sequencing comprises whole-exome sequencing.

17. A method for inhibiting a variant Btk, comprising contacting the variant Btk with an effective amount of a compound of formula (I): 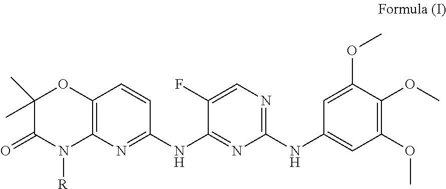
wherein R is hydrogen, —CH₂OP(O)(O⁻)₂X⁺_{2,} or —CH₂OP(O)(O⁻)₂Y²⁺, and each X is independently a hydrogen ion or a monovalent cation, and Y²⁻ is a divalent cation;
or a pharmaceutically acceptable salt, hydrate or solvate thereof.

18. The method of claim 17, wherein the variant Btk is a Cys481 variant.

19. The method of claim 17, wherein the variant Btk is contacted in a cell.

20. The method of claim 19 wherein the cell is a B-cell.

21. The method of claim 19, wherein the cell is a lymphoma cell.

22. The method of claim 17, wherein the compound is of Formula (Ia): 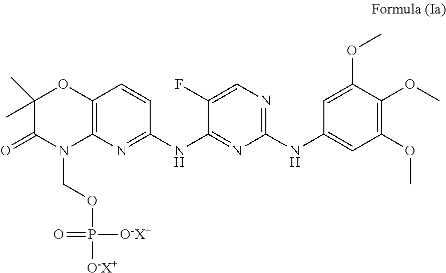
wherein each X⁺ is an alkali metal cation;
or a hydrate or solvate thereof.

23. The method of claim 17, wherein the compound is Compound (I): 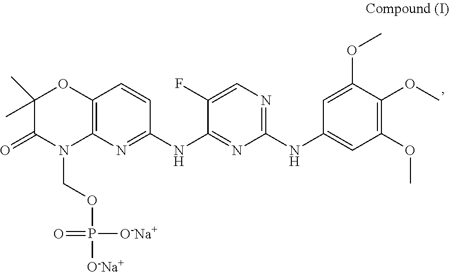
or a hydrate or solvate thereof.

24. The method of claim 17, wherein the compound is of Formula (Ib): 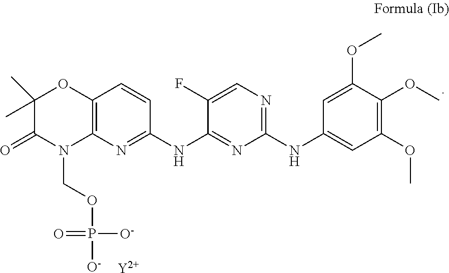
wherein the divalent cation Y²⁻ is an alkali earth metal;
or a hydrate or solvate thereof.

25. The method of claim 17, wherein the compound is Compound (II): 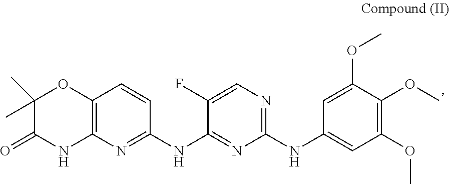 [SMILES: COc1cc(Nc2ncc(F)c(Nc3ccc4c(n3)NC(=O)C(C)(C)O4)n2)cc(OC)c1OC]
or a pharmaceutically acceptable salt, hydrate or solvate thereof.

26. The method of claim 17, further comprising contacting the variant Btk with a second Syk inhibitor.

27. The method of claim 17, further comprising first identifying a cell with a variant Btk.
